(19) United States
(12) Patent Application Publication
(10) Pub. No.: US 2021/0388058 A1
(43) Pub. Date: Dec. 16, 2021

(54) METHOD FOR MANUFACTURING HIGHLY PURIFIED LACTOFERRIN AND LACTOPEROXIDASE FROM MILK, COLOSTRUM AND ACID OR SWEET WHEY

(71) Applicant: ARHEL PROJEKTIRANJE IN INZENIRING D.O.O., Ljubljana (SI)

(72) Inventors: Marko KETE, Ajdovscina (SI); Blaz LOKAR, Ajdovscina (SI); Maja Zupancic JUSTIN, Vrhnika (SI); Ales STRANCAR, Ajdovscina (SI)

(21) Appl. No.: 17/291,081

(22) PCT Filed: Nov. 6, 2019
(86) PCT No.: PCT/EP2019/080428

§ 371 Date: May 4, 2021

(30) Foreign Application Priority Data
Nov. 6, 2018 (EP) 18204674.8

(51) Int. Cl. C07K 14/79; C12N 9/08

(52) U.S. Cl. CPC C07K 14/79; C12Y 111/01017; C12N 9/0065

(57) ABSTRACT

A method for manufacturing a fraction comprising the proteins lactoferrin and/or lactoperoxidase from a source containing at least one of these proteins wherein the source is selected from the group consisting of milk, colostrum, acid or sweet whey, by means of a chromatographic separation process with a monolithic column having cation exchanger properties, wherein in the separation process a pH gradient or a combined pH and salt gradient elution is employed after loading the source to the column.

Further disclosed is a composition of matter comprising lactoferrin having a C value of >60% and A value of >1% or lactoperoxidase.

The invention pertains to a method for manufacturing a fraction comprising the lactoferrin or lactoperoxidase proteins from a source containing at least one of these proteins and the highly purified proteins lactoferrin or lactoperoxidase.

### INTRODUCTION

Lactoferrin (LF) and Lactoperoxidase (LPO) are functional minor proteins present in milk, whey and colostrum. LF is an 80 kDa glycosylated protein that can respond to a variety of physiological and environmental changes and is therefore considered a key component in the host's first line of defense. The structural characteristics of LF provide functionality in addition to the Fe³⁺ homeostasis function, common to all transferrins: strong antimicrobial activity against a broad spectrum of bacteria, fungi, yeasts, viruses and parasites; anti-inflammatory and anticarcinogenic activities; and several enzymatic functions [1]. LPO plays a vital role in protecting the lactating mammary gland, the intestinal tract of new born infants against pathogenic microorganisms, is involved in the degradation of various carcinogens and the protection of animal cells against peroxidative effects [2].

LF is able to bind iron. Native LF comprises 15 to 20% of holo LF, which contains iron. The remaining part is apo LF, which does not contain iron [32]. In theory, apo LF has a rather low iron saturation level (already bound iron—A value). The theoretical A value of apo LF is about <3%. Additionally, apo LF possesses a high potential for iron binding (iron capacity—C value). The C value of apo LF is about >50%. Highly pure and non-denatured apo LF has a potential to have even higher C values of >70%. On the other hand, holo LF possesses a high A value (>50%) and a low C value (<10%). The higher the C value of isolated LF, the higher the iron binding potential of LF. A higher potential of binding iron leads to a higher level of antimicrobial activity, because the active LF removes essential iron required for microbial growth. Nowadays LF and LPO are isolated from milk and milk processing byproducts (e. g. whey) by many different techniques like: (I) isolation by paramagnetic particles with poly(glycidylmethacrylate) with heparin ligand [3], (II) by using of cationic surfactant (e. g. Cetyldimethylammonium bromide) [4], (III) different chromatographic techniques (e. g. cation exchange or affinity chromatography) [1,2,5-10] and (IV) other techniques (e. g. hydrophobic ionic liquids [11]). In general, chromatographic techniques, most of all the ion exchange chromatography, represent a way how to separate LF rapidly at relatively low costs [12]. The chromatographic approach also prevails over others due to its robustness and repeatability. The most common technique in the chromatographic purification of LF and LPO is using strong cation exchange resin particles and membranes or monolith columns [13-15].

The present chromatography suffers from some drawbacks: (I) peak broadening with increasing flow velocity due to the diffusion mass transfer in the pores, so when the flow velocity is increased, the gradient slope must become shallower in order to obtain the same resolution, (II) increased flow decreases time of chromatographic step but at the same time increases elution volume and (III) column length results in high column pressure drop, which is a limiting factor for flow velocity.

In chromatographic purification step LF and LPO present in milk or whey are under certain conditions bound to surface of strong cation exchanger and afterwards collected in an elution fraction using buffers with higher pH or high salt concentration. To simplify the separation process, LF and LPO are most often eluted using several buffer solutions with either high ionic strength or pH>9 in a step mode. Such approach often results in relatively high purity (60-95%) of the fraction of desired protein in one chromatographic step. To desalt, concentrate and increase the purity, an ultrafiltration process is often introduced to eliminate the slight amount of low-molecular-weight impurities. The obtained protein concentrate is then usually dried by lyophilisation or spray drying. Wang et al. [18] have shown that lyophilised LF contains less water (approx. 2-3%) than spray-dried (≈5%), while on the contrary, spray-dried LF showed a slightly lower degree of denaturation and 6-7% higher antioxidant activity, which was close to that of fresh liquid LF. Spray drying [1], is confidently used to produce amorphous LF powders with intact molecular configuration and high antioxidant capacities.

EP 0418704 A1 [19] describes processes of separating, purifying and recovering milk proteins capable of binding iron using ion exchange chromatographic columns, which contain resin particles with surface sulfonic groups. LF is being isolated by pH/conductivity step elution mode, and the final product purity is claimed to be >90%. Also, an ultrafiltration process is required to eliminate the slight amount of low-molecular-weight impurities, ultimately rendering LPO and LF of 90% or higher purity.

The process described in EP 1466923 A1 [20] includes a chromatographic step with strong-acid cation-exchange resin (particles), where purity of isolated LF is in a range between 79 and 91%.

WO 2006/119644 A1 [21] describes a method for purifying LF, stabilising it in solution and improving its activity. The process is intended for additional purification of already isolated LF of lower purity. Purification is conducted using a hydrophobic adsorbent (particles) in the presence of an aqueous acidic solution containing a concentration of a charged excluded solute. The final purity of LF achieved was >95%.

U.S. Pat. No. 5,861,491 A [13] discloses methods for isolating human LF, including human LF produced by expression of a transgene encoding a recombinant human LF (rhLF), as well as other related LF species from milk, typically from bovine milk. In general, milk or a milk fraction containing hLF is contacted with a strong cation exchange resin in the presence of relatively high ionic strength to prevent binding of non-LF proteins and other substances to the strong cation exchange resin. Resin particles are afterwards separated from milk by centrifugation and LF bound to the cation exchange resin is then eluted using few buffer solutions with a different salt concentration in a step mode. The purity of top fractions of hLF and bLF exceeds approximately 95%.

EP 0348508 B1 [22] discloses the LF isolation from raw milk using a sulfonated crosslinked polysaccharide resin (particles).

LF adsorbed on the column is eluted in step mode using a buffer with increased NaCl concentration. The purity of the bovine LF was measured at 95% while LF obtained from defatted human colostrum was 98% pure.

The method described in U.S. Pat. No. 6,096,870 A [23] is related to the separation of whey proteins (immunoglobulin, β-lactoglobulin, α-lactalbumin, bovine serum albumin, LF), particularly the sequential separation of whey proteins into separate fractions using a prepacked chromatographic column with strong cation exchange resin particles. Sequential elution of mentioned protein fractions is achieved with buffers at suitable pH and ionic strength in a stepwise mode. Final spray dried products purity was: immunoglobulins ≥80%, BSA and LF ≥75% and β-lactoglobulin ≥85%.

U.S. Pat. No. 8,603,560 B2 [24] describes a process for isolating milk proteins from milk or whey using cation-exchange resin packed in a column. Elution is promoted by a stepwise change of pH or ionic strength. Final LF purity was 80%.

CA 2128111 C [25] describes the process for isolating LF and LPO from milk and milk products on an industrial scale. Isolation is achieved by adsorbing said proteins to a cation exchanger and eluting these proteins separately or simultaneously, by step elution with one or more salt solutions. There is no data on the final purity of isolated proteins.

EP 0253395 B9 [26] discloses a process for isolation of bovine LF from milk in high purity using weakly acidic cation-exchange resin. LF is recovered from ion exchanger by stepwise elution with sodium chloride solutions having different concentrations. According to the old Laurell's method, the purity of produced LF is measured to be up to 90-99%.

U.S. Pat. No. 5,596,082 A [27] discloses a process for isolating the LF and the enzyme LPO from milk and milk products on an industrial scale. The process includes the steps of adsorbing these proteins to a cation exchanger by passing milk or milk derivatives over the cation exchanger. Elution of mentioned proteins is promoted by step elution using different salt concentration. Final purity of LPO and LF was up to 93% and 94%, respectively.

The invention disclosed in U.S. Pat. No. 9,115,211 B2 [28] describes an isolation of LF using cation exchange resin. LF obtained by described method is more than 95% pure, substantially free of LPS, endotoxins and angiogenin with an iron saturation level comprised between 9% to 15%. EP2421894A1 [29] describes a method of preparing low-iron LF with less than 10% iron saturation or, more preferably about 9% to 3.89% iron saturation. This low iron LF produced by the process shows an increased antimicrobial activity in comparison to standard LF. This process uses acid and solvent. After Fe³⁺ released the process aids added were removed by UF and DF process. The resulting product is a light cream/pale beige colour with 3.89% to 5.1% iron saturation (by HPLC/X-ray fluorescence (XRF)).

WO2014/207678 A1 [30] discloses a method of purifying LF from a secretory fluid, the method comprising alkalizing the secretory fluid, contacting the alkalized secretory fluid with air, and precipitating LF from the alkalized secretory fluid using an organic solvent (acetone). WO1995/022258 A2 [31] discloses methods for purification of human LF from milk, especially milk of nonhuman species, and for separation of human LF from undesired macromolecular species present in the milk, including separation from nonhuman LF species. To perform the isolation, strong cation exchange resin (e.g., S Sepharose™) is used. Proteins (LF and others) were eluted with a stepped salt and pH gradient.

G. Majka et al. (2013) A high-throughput method for the quantification of iron saturation in lactoferrin preparations, Anal. Bioanal. Chem., 405, 5191-5200 discloses a method for obtaining bovine lactoferrin with low iron content. It was not sufficient to perform ion exchange chromatography to obtain lactoferrin with low iron content. Therefore, ion exchange chromatography was combined with extensive dialysis against 100 mM citrate buffer for 24 h.

### SUMMARY OF THE INVENTION

One object of the invention is to provide a composition of matter of highly active lactoferrin with high purity.

Another object of the invention is to provide a method appropriate to overcome at least some of the disadvantages of the prior art.

The highly active lactoferrin is characterised by its rather low iron saturation level (already bound iron—A-value) and its high potential for iron binding (iron binding capacity—C-value). Another object of the invention is to provide a composition of matter comprising lactoperoxidase in high purity.

Yet another object of the invention is to provide for a method of manufacturing lactoferrin or lactoperoxidase in high purity from these proteins containing sources.

These objects are solved by a method for manufacturing a fraction comprising the proteins lactoferrin or lactoperoxidase from a source containing at least one of these proteins, wherein the source is selected from the group consisting of milk, colostrum, acid or sweet whey, by means of a chromatographic separation process with a monolithic column having strong cation exchanger properties, wherein in the separation process a pH gradient elution or a combined pH and salt gradient elution is employed after loading the source to the column.

In one embodiment of the invention the pH gradient starts typically in a pH range of 4.0 to <pH 8.0, preferably in a pH range of 4.0 to 7.5, more preferably in a pH range of about 4.0 to about <pH 7, in particular in a pH range of about 4.5 to about <pH 6.5.

In another embodiment of the invention the pH gradient terminates typically in a range of about pH 8 to pH<13, preferably in a pH range of pH 8 to pH 12, in particular in a pH range of pH 8 to pH<12.

It can be advantageous to filter the source prior to loading of the source to the column.

In still another embodiment of the invention the salt gradient is performed by increasing the salt concentration, in particular the salt gradient corresponds to a conductivity in a range of about 5 mS/cm to about 55 mS/cm. It is recommendable to use neutral salts for adjusting the salt concentration in order to avoid interference with the pH of the buffer solution. In particular useful are salts which are employed in processes of the food industries, typically sodium chloride. The pH gradient used in combination with the salt gradient mentioned before starts with a pH value typically in a pH range of 4.0 to <pH 8.0, preferably in a pH range of 4.0 to 7.5, more preferably in a pH range of 4.0 to <pH 7, in particular in a pH range of 4.5 to <pH 6.5. The pH gradient used in combination with the salt gradient terminates typically in a range of pH 8 to pH<13, preferably in a pH range of pH 8 to pH 12, in particular in a pH range of pH 8 to pH <12.

In yet another embodiment of the invention a fraction A can be collected which elutes at a pH range of about pH 8 to about pH<11, preferably at a pH range of 8.0 to pH 10.0, more preferably at a pH range of pH 8.2 to pH 10.0, in particular about pH 8.9 to about pH 10. This fraction contains typically lactoperoxidase.

In still another embodiment of the invention a fraction B can be collected which elutes at a pH range of >10 to pH 12.0, preferably at a pH range of pH >10.4 to pH 12, preferably about pH >11 to about 12, in particular about pH >11 to about 11.7. This fraction contains typically lactoferrin.

In a particularly useful embodiment of the method of the invention the chromatographic separation process comprises the steps of
(i) Adjusting the pH value of the source to a value lower than pH 7, in particular lower than pH 6.5;
(ii) Contacting the source of step (i) with a monolithic column having strong cation exchanger properties; followed by
(iii) Flowing a gradient buffer through the column thereby increasing the pH value; and
(iv) Collecting a fraction A which elutes at a pH range of about pH 8 to about pH<11, preferably at a pH range of pH 8.0 to pH 10.0, preferably at a pH range of pH 8.2 to pH 10.0, in particular about pH 8.9 to about pH 10; and/or
(v) Collecting a fraction B which elutes at a pH range of >10 to pH 12.0, preferably about pH >10.4 to about 12, preferably at a pH range of pH >11.0 to 12.0, in particular about pH >11 to about 11.7;
(vi) Optionally further processing the fractions A and/or B, in particular by treatment for neutralising, concentrating, preservation and the like.

It can be useful to filter the source containing the lactoferrin and/or lactoperoxidase prior to step (ii).

In another embodiment of the method of the invention the monolithic column can be equilibrated prior to step (ii) with an equilibration buffer having a pH value of about pH<7, in particular about pH<6.

The monolithic column having strong cation exchanger properties in particular is selected from the group consisting of a SO₃H modified monolithic column, —COOH modified monolithic column, —OSO₃H modified monolithic column or —OPO₃H modified monolithic column. In the context of the invention SO₃H, —COOH, —OSO₃H, or —OPO₃H modified monolithic column also encompasses the corresponding salts of the acidic moieties, in particular their alkali salts such as sodium, potassium salts for example SO₃Na, —COONa, —OSO₃Na, or —OPO₃Na or SO₃K, —COOK, —OSO₃K, or —OPO₃K.

According to the method of the invention fraction A, eluting at a lower pH value than fraction B, contains typically lactoperoxidase whereas fraction B contains typically lactoferrin. In a further embodiment of the invention the column can be flushed with the equilibration buffer prior to step (iii) or (iv).

Typically, the lactoferrin and lactoperoxidase containing fractions can be further processed for example dried, in particular by spray drying.

The method of the invention yields lactoferrin or lactoperoxidase of high purity. The purity of lactoferrin is >98% and the purity of lactoperoxidase is >78%. Furthermore, the lactoferrin C value is >50% or >60% and the lactoferrin A value is >1%. Preferably, the lactoferrin C value is >70% and the lactoferrin A value is >2%. Preferably, the lactoferrin C value is >70.0%, preferably between 70.0% to 80.0%, more preferably between 70.0% and 77.0%. Preferably, the lactoferrin A value is >2.0%, preferably <3.9%, preferably between 1.0% and 7.0%, preferably between 2.0% and 7.0%, preferably between 2.0% and 5.0%, more preferably between 2% and 4%.

According to another embodiment of the invention the monolithic column can be sanitised by flushing with a buffer of about pH >12, typically after step (iv) or (v).

Subject matter of the invention is also a composition of matter comprising lactoferrin or lactoperoxidase obtainable by the method according to the invention. The C value of lactoferrin is >60% and the A value is >1%. Preferably, the C value of lactoferrin is >70% and A value of >2%. Preferably, the lactoferrin C value is ≥70.0%, preferably between 70.0% to 80.0%, more preferably between 70.0% and 77.0%.

Preferably, the lactoferrin A value is between 1.0% and 7.0%, preferably between 2.0% and 7.0%, preferably between 2.0% and 5.0%, more preferably between 2% and 4%, preferably is ≥2.0%, and/or preferably <3.9%.

Preferably the A+C value of the product of the present invention is at least 61%, or at least 72% or at least 73%.

The method of the invention provides for
a rapid and simple method for LF and LPO isolation from milk, colostrum and acid or sweet whey,
a single chromatographic step isolation process using pH linear gradient for LF and LPO isolation, which results in highly pure target proteins (e.g. LF purity >98%),
a product (LF protein) obtained by the disclosed method which is, according to a well-established method for measuring Unsaturated Iron Binding Capacity (UIBC), highly active (C-value >70%, C-value determination kit for LF, NRL Pharma) and contains low amount of already bound iron (A-value <3.9%, A-value determination kit for LF, NRL Pharma), which are leading characteristics of LF available on the market. The product is produced by an economical and procedurally undemanding method without using special chemicals and is conducted on site (on monolith cation exchanger),
a method, which enables production of LF which contains low amount of iron (A-value <3.9%) and is at the same time free of angiogenin,
a method, which enables to produce LF with high total bioactivity (C-value+A-value>72%),
a method which can easily be scaled-up to a production scale and allowing cost-effective production of highly pure and bioactive LF and LPO,
a whole process of LF/LPO isolation comprising chromatography, concentration/desalting and drying process that provides overall >85% LF recovery,
a chromatographic process that allows high flow rates of mobile phase (700 L/m²/h) without impeding high resolution of proteins resulting in high productivity process,
a process that does not introduce chemicals into the media with proteins intended for isolation (e. g. whey) or otherwise changes its characteristic; this allows further use of media in other biochemical industrial processes,
a method enabling the production of LF with pre-defined share of already bound Fe.

### BRIEF DESCRIPTION OF THE FIGURES

FIG. 1. Chromatograph of LF elution peak, composed from LF elution subpeaks. The phenomenon is a consequence of small differences in LF isoelectric point (IEP) due to its iron content. Voswinkel et al. [32] showed, that decrease in iron content consequently lowers IEP of LF to a small degree.

FIG. 2. Chromatographs of (A) acid whey and (B) commercial LF products and LF.

FIG. 3. Scheme of LF isolation from acid whey using one 8 L monolith column, CIMmultus™ SO3; BIA Separations and basic information on the mass balance of the process.

### DETAILED DESCRIPTION OF THE INVENTION

Monolithic columns with strong cation exchanger groups CIMmultus™ SO3—strong CEX from BIA Separations were tested in different elution modes to isolate LF and LPO from filtered whey or milk separately. Loading of LF/LPO was performed at natural pH of whey, while the two target proteins were eluted separately by (I) stepwise increase of the pH, (II) conductivity steps, (III) linear conductivity gradient and (IV) linear pH gradient.

In the case of step elution modes, performed for comparison, the purity of LF achieved was about 95%, while in the case of conductivity gradient the purity achieved was slightly higher than 95%. Surprisingly, it has been found that according to the invention an elution with an increasing pH gradient, in particular with a linear increasing pH gradient, the protein purity achieved was over 98%. The purity was proven by HPLC, SDS-page analysis, Bioanalyzer and SEC chromatography. The method of the invention provides for these results by a single chromatographic step and on a production scale. By the way, according to Laurell's method [26] used for purity calculation in EP 0253395, the calculated purity for the product of the invention which is obtainable by the method of the invention is >118%. It seems that the established method becomes outdated and is not suitable any more for the correct determination of LF purity.

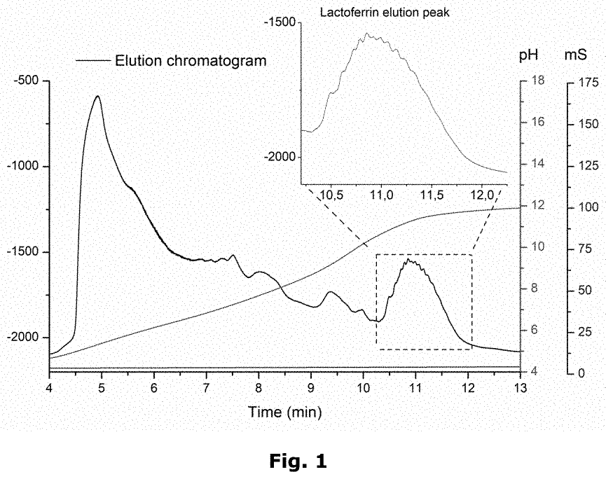

In the case of linear pH gradient elution, it was noticed that LF elution was composed of several smaller sub-peaks (FIG. 1). The latter suggests that LF was also separated on the level of protein iron content, from lower to higher. The reason for this was already discussed and proven by others [32].

Concentrated LF water dispersion was dried by spray drying or lyophilizer, and unsaturated iron-binding capacity (UIBC) for LF was than measured. According to Iron Colorimetric Assay Kit provided by NRL Pharma Inc. Japan (detailed principle was published by Ito et al. [33]), LF obtained by the method of the invention had an iron saturation level (already bound iron—A value) between 2% to 4.9%. Its potential for iron binding (iron binding capacity—C value), also called unsaturated iron-binding capacity (UIBC), was above 70%. The result ranks at the top in comparison to products present on the market, whose A and C values rank between 4.6-11.7% and 34.4-52.1%, respectively (see Table 1). At the same time their total bioactivity (C value+A value) is usually lower than in comparison to the product of the invention.

TABLE 1
Bioactivity of commercially available LF samples
and LF of the invention (Arhel d.o.o.)

| Samples of LF | C value [%] | A value [%] | (A + C)* [%] |
|---|---|---|---|
| Supps Planet | 34.4 | 4.6 | 39.0 |
| Lactoferrin | | | |
| Life Extension | 48.6**(31.1-vz.) | 11.7**(7.5-vz.) | 60.3 |
| (Lactoferrin | | | |
| caps-Bioferrin; | | | |
| 95% | | | |
| Apolactoferrin) | | | |
| Ingredia | 52.1 | 7.7 | 59.8 |
| Nutritional | | | |
| (Prodiet | | | |
| Lactoferrin, | | | |
| >95%) | | | |
| NRL Pharma | 49.2 | 9.0 | 58.2 |
| LF of the | 70.0-74.0 | 2.0-4.9 | 74.9 (avg.) |
| invention | | | |

*The total bioactivity (A + C) is expressed by summing the C and A values, which gives the percentage of active protein in the sample.
**Value is recalculated according to the quantity of pure LF in the sample, which is 64%.

The method of the invention provides a method for manufacturing a fraction comprising the proteins lactoferrin or lactoperoxidase from a source containing at least one of these proteins, wherein the source is selected from the group consisting of milk, colostrum, acid or sweet whey, by means of a chromatographic separation process with a monolithic column having strong cation exchanger properties, in particular a —SO₃H modified monolithic column wherein in the separation process a pH gradient elution is employed after loading the source to the column. A monolithic column is commonly a chromatographic separation equipment comprising a hollow body wherein a porous solid material is contained which is a polymerisate of monomers. Pores of the material are formed e.g. during the polymerisation process (U.S. Pat. Nos. 4,923,610, 4,952,344, 4,889,623).

Suitable devices are described in the prior art, for example EP 1058844, EP 777725 and are commercially available. The monolithic chromatography material used herein is modified with —SO₃H moieties which are exposed i. a. at the surfaces of the porous material. The surface modification with —SO₃H groups provides the material with so called strong cation exchanging properties (CAX). The skilled person knows that other materials, e. g. classified as weak cation exchanger. In contrast to this, for other purposes anion exchanger (AEX) can be used.

A pH gradient chromatography is conducted by increasing the pH from a starting value to an end point. It can be designed in an almost linear shape but also a different course is possible as long as the result of the invention, i. e. the products lactoferrin and/or lactoperoxidase of the invention are obtained. The skilled person knows how to perform the gradient chromatography as such. An optimisation of the conditions of the cationic gradient chromatography—based on the explicit and implict disclosure of the invention-lies within skilled person's routine and is not related with undue burden of experimentation.

In order to establish conditions for a reproducible processing it can be advantageous to equilibrate the monolithic column prior to the actual separation by pH gradient. In this case the column is flushed with an equilibration buffer. Typically, the column is flushed with a volume of the equilibration buffer equivalent to 8-12× dead volumes of the column. The selection of the starting pH value of the chromatography is to some extent influenced by the pH value of the source containing LF and/or LPO. For example, if acid whey is the source for LF and/or LPO, the pH value for equilibration can be pH 4.6 whereas if sweet whey is used, the pH value can be higher, i. e. pH 5.0 to pH 6.5.

It can be advantageous to filter the source prior to loading of the source to the column. Generally, filter means used in the diary industries can be employed. Particularly useful are ceramic TFF filters, spiral-wound membranes or other continuous filtration technologies. After loading the source on the monolithic column, in principle, the pH gradient chromatography can start. It may be useful, however, that prior to starting the pH gradient chromatography, a further flushing of the column with a pH value around the equilibration conditions can be employed to remove impurities. This supports the separation of the proteins to be manufactured, because proteins or other contaminants which elute at that pH value do not pollute the separation of LF und/or LPO.

The pH gradient starts with a pH value typically in a pH range of 4.0 to <pH 8.0, preferably in a pH range of 4.0 to 7.5, preferably in a pH range of about 4.0 to about <pH 7, in particular in a pH range of about 4.5 to about <pH 6.5. The pH gradient terminates typically in a range of about pH 8 to pH<13, preferably in a pH range of pH 8 to pH 12, in particular pH 8 to pH<12. FIG. 1 depicts a typical course of a pH gradient chromatography.

It should be noted that also the ionic strength of the elution buffer can have some influence on the separation. The ionic strength can also follow an increasing gradient during the chromatographic separation overlaying the necessary pH gradient. The necessary pH gradient for use in combination with the salt gradient mentioned before starts with a pH value typically in a pH range of 4.0 to <pH 8.0, preferably in a pH range of 4.0 to 7.5, preferably in a pH range of 4.0 to <pH 7, in particular in a pH range of 4.5 to <pH 6.5. The pH gradient used in combination with the salt gradient terminates typically in a range of pH 8 to pH<13, preferably in a pH range of pH 8 to pH 12, in particular in a pH range of pH 8 to pH<12.

For example LPO is eluting at a pH range of about pH 8 to about pH<11 if the ionic strength is equivalent to a conductivity of about 15 mS/cm, but at lower pH in the range of about pH 6.6 to about pH 7.5 if the conductivity increases from 4 to 55 mS/cm, preferably from 5 to 55 mS/cm. The elution of LF follows a similar regime. If the conductivity is medium but stays constant, LF elutes in a range of about pH 10.7 to about pH 11.7, if the conductivity increases from 4 to 55 mS/cm, preferably from 5 to 55 mS/cm, LF is eluting at lower pH in the range of about pH 9.6 to about pH 10.7. The ionic strength, i. e. conductivity can be adjusted by adding suitable salts. Using suitable salts also the pH value of the elution buffer may be adjusted. A fraction A eluting at a pH range of about pH 8 to about pH<11, preferably at a pH range of pH 8.0 to pH 10.0, preferably at a pH range of pH 8.2 to pH 10.0, in particular about pH 8.9 to about pH 10, or about pH 6.6 to about pH 7.5 at higher conductivity about 5 to 55 mS/cm is collected. This fraction contains typically lactoperoxidase. A fraction B eluting at a pH range of >10 to pH 12.0, preferably at a pH range of pH >10.4 to pH 12, preferably about pH >11 to about 12, in particular about pH >11 to about 11.7, or about pH 9.6 to about pH 10.7 (higher conductivity, about 5 to 55 mS/cm) is collected. This fraction contains typically lactoferrin. In the following a typical performance of the method of the invention is described. The pH ranges where LF and LPO are eluting correspond to a medium conductivity of about 15 mS/cm. The chromatographic separation process comprises the steps of

(i) Adjusting the pH value of the source to a value lower than pH 7, in particular lower than pH 6.5;
(ii) Contacting the source of step (i) with a monolithic column having strong cationic properties, in particular a —SO₃ modified monolithic column; followed by
(iii) Flowing a gradient buffer through the column thereby increasing the pH value; and
(iv) Collecting a fraction A which elutes at a pH range of about pH 8 to about pH<11, preferably at a pH range of pH 8.0 to pH 10.0, preferably at a pH range of pH 8.2 to pH 10.0, in particular about pH 8.9 to about pH 10; and/or
(v) Collecting a fraction B which elutes at a pH range of >10 to pH 12.0, preferably about pH >10.4 to about 12, preferably at a pH range of >11 to pH 12, in particular about pH >11 to about 11.7;
(vi) Optionally further processing the fractions A and/or B, in particular by treatment for neutralising, concentrating, preservation and the like.

In order to remove unwanted material, the source containing the lactoferrin and/or lactoperoxidase is filtered prior to step (ii) through a ceramic filter.

The monolithic column is equilibrated prior to step (ii) with an equilibration buffer having a pH value of about pH<7, in particular about pH<6. The column is flushed with the equilibration buffer prior to step (iii) or (iv).

After collecting the lactoferrin and lactoperoxidase containing fractions these are further processed by spray drying.

The obtained lactoferrin or lactoperoxidase is of high purity. The purity of lactoferrin is >98% and the purity of lactoperoxidase is >78%. Furthermore, the lactoferrin C value is ≥60% and the lactoferrin A value is ≥1%. Preferably, the lactoferrin C value is >70% and the lactoferrin A value is >2%. Preferably, the lactoferrin C value is ≥70.0%, preferably between 70.0% to 80.0%, more preferably between 70.0% and 77.0%. Preferably, the lactoferrin A value is preferably between 1.0% and 7.0%, preferably between 2.0% and 7.0%, preferably between 2.0% and 5.0%, more preferably between 2% and 4%, preferably ≥2.0% and/or preferably <3.9%,

According to another embodiment of the invention the monolithic column can be sanitised by flushing with a buffer of about pH >13, typically after step (iv) or (v).

A sanitising step is performed by flushing column with deionized water (10-15 CVs), 1M NaOH (4-10 CVs) with contact time of 1 to 3 h and again flushing with water (>30 CVs). This step may be executed every 8-10 chromatographic runs.

The uniqueness of the novel approach according to the method of the invention is also in the facts that it (I) does not require any chemical modification of the source (e.g. whey) from which the protein is isolated, but merely pre filtration using standard filtration techniques, (II) in contrast to other processes (e.g. EP2421894 A1 [29]) uses inexpensive chemicals (buffers) in low amounts, (III) provides separate highly pure fractions of LPO and LF, (IV) enables the production of LF with pre-defined share of already bound Fe and (V) results in high protein recovery, which is higher than 85%, including desalting/concentrating of protein elution dispersion and drying production steps.

All references cited herein are incorporated by reference to the full extent to which the incorporation is not inconsistent with the express teachings herein.

The invention is further explained and illustrated in and by the following non-limiting examples.

### EXAMPLES

Analytics:

HPLC Analytics:

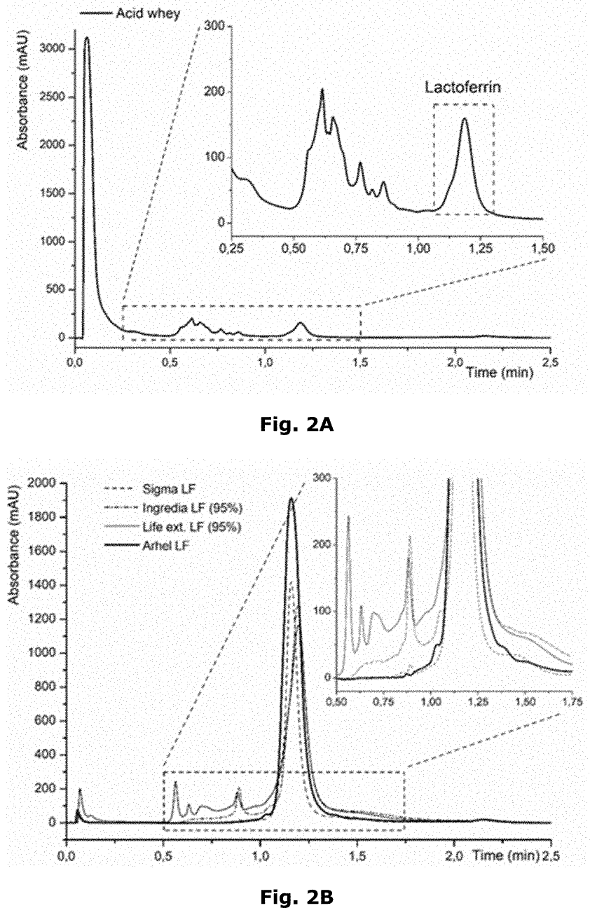

To determine LF and LPO in samples, a chromatographic system equipped with MWD multi-wavelength detector and conductivity monitor with pH measuring kit (PATfix™, BIA Separations d.o.o., Ajdovscina, Slovenia) was employed. Chromatographic separations were run on CIMac™ S03-0.1 (Pores 1.3 μm, BIA Separations d.o.o., Ajdovscina, Slovenia) analytical column using two sodium phosphate monobasic buffer solutions (25 mM, pH=7.5) in conductivity gradient mode which increased from 3.5 to 152 mS/cm. The elution of analysed proteins were monitored by MWD detector at 226 nm. Detailed information about the chromatographic method is presented in Table 2, while chromatograms of some analysed samples are shown in FIGS. 1 and 2. All samples were prior analysis filtered through CHROMAFIL® A-45/25, 0.45 μm, cellulose mixed esters filters.

TABLE 2
Chromatographic system and method used for sample analysis.
Chromatographic system: PATfix ™ HPLC system
Chromatographic column: CIMac ™ SO3, 100 μL (BIA Separations d.o.o., Ajdovscina, Slovenia)
Injection volume: 15 μL
Detection: 226 nm

| | | B (%) | |
|---|---|---|---|
| | A (%) | (c(NaH₂PO₄ × 2H₂O) = | |
| Time | (c(NaH₂PO₄ × 2H₂O) = | 25 mM, c(NaCl) = | Flow |
| (min) | 25 mM, pH = 7.5) | 2M, pH = 7.5) | (mL/min) |
| 0 | 100 | 0 | 2.6 |
| 0.2 | 100 | 0 | 2.6 |
| 1.24 | 0 | 100 | 2.6 |
| 1.85 | 0 | 100 | 2.6 |
| 1.87 | 100 | 0 | 2.6 |
| 3.0 | 100 | 0 | 2.6 |

Determination of C- and A-Values:

To determine C- and A-values of dry LF samples we used Iron Colorimetric Assay Kit provided by NRL Pharma Inc., Japan (detailed principle was published by Ito et al. [33]), while to determine A-value for dry and liquid samples we used the same approach as described in literature [33,34] in combination with HPLC method.

Determination of C Values:

To determine the C value of LF, LF was saturated by a known excess amount of iron. The remaining iron was coloured by a chelating reagent (maximum absorbance 760 nm). The remaining iron is quantified spectrophotometrically at 760 nm. A serial dilution of the iron complex is prepared and a calibration curve is determined spectrophotometrically at 760 nm. The bound iron is calculated by subtracting the remaining iron from the added iron. The C value is indicated in relative terms, wherein the calculated theoretical iron binding capacity of LF (each molecule of LF can bind 2 iron atoms) is set as 100%.

Calculation of the C Value:

$$theoretical iron binding capacity ( LF ) [ \frac{μ g}{dl} ] = \frac{amount of LF}{volume of sample} \times \frac{M ( Fe )}{M ( LF )} \times \frac{1000 μ l \times 100000 μ l}{1 mg \times 1 dl} \qquad ( 1 )$$

M(Fe): molecular weight of iron; M(LF): molecular weight of LF

$$iron bound by LF [ \frac{μ g}{d l} ] = c ( {Fe}_{Start} ) - c ( {Fe}_{End} ) \qquad ( 2 )$$

c(Fe_{Start}): starting concentration of iron; c(Fe_{End}): concentration of remaining iron

$$C value [ \% ] = \frac{iron bound by LF}{theoretical iron binding capacity LF} \times 100 \qquad ( 3 )$$

Determination of a Values:

To determine the A value of LF, LF was denaturated by a denaturating reagent and the released iron was coloured by a chelating agent (maximum absorbance 760 nm). The released iron is quantified spectrophotometrically at 760 nm. A serial dilution of the iron complex is prepared and a calibration curve is determined spectrophotometrically at 760 nm. The already bound iron (A value) is indicated in relative terms, wherein 2 iron atoms bound to one LF molecule is defined as 100%.

Calculation of the a Value:

$$( LF ) [ \frac{μ g}{d l} ] = \frac{amount of LF}{volume of sample} \times \frac{M ( Fe )}{M ( LF )} \times \frac{1000 μ l \times 100000 μ l}{1 mg \times 1 dl} \qquad ( 1 )$$

M(Fe): molecular weight of iron; M(LF): molecular weight of LF

$$A value [ \% ] = \frac{iron released from LF}{theoretical iron binding capacity LF} \times 100 \qquad ( 4 )$$

Scheme of A Pilot System

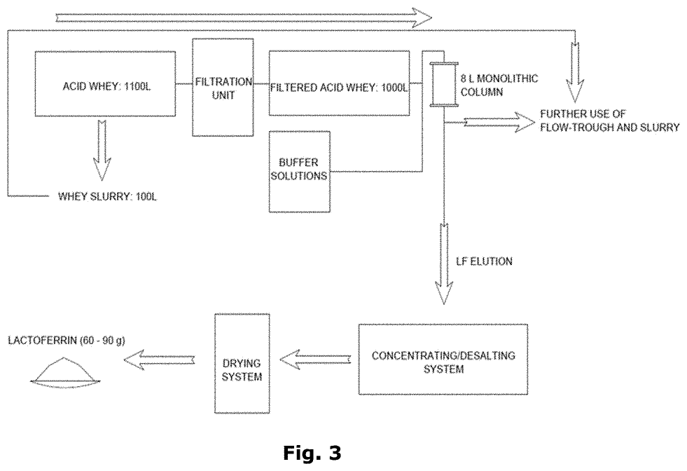

FIG. 3 depicts a flow sheet about the principles in the technology for LP/LPO isolation and approximate values on the mass balance of the process on a scale of one chromatographic run. Before LF/LPO isolation, 1,100 L of acid whey is filtered using ceramic TFF filter system with a pore diameter lower than 0.8 μm. Filtered whey (1,000 L) is then pumped trough an equilibrated chromatographic column. The column-bound LF and LPO are after that eluted by a combination of different buffer solutions. Eluted fractions are then concentrated and, if necessary, desalted. After drying of concentrated protein, 60 to 90 g of dry LF product are obtained. Negligibly changed flow trough whey and whey slurry can be, separately or mixed, further used or processed downstream.

### Example 1

A monolith column, 80 mL CIMmultus™ SO3; BIA Separations was before loading equilibrated using 800 mL of buffer solution A (sodium phosphate or citrate buffer: 5-50 mM, pH =4.6). After the equilibration, filtered acid whey was pumped through the column until the column capacity for LF and LPO were reached. The saturation of the column capacity was verified by analysing flow-through samples on the outflow side of the column by the HPLC method described in the Analytics section. The volume of whey pumped through the column at a flow rate of 0.24 L/min was usually 10 to 20 L, which was mainly depending on LF/LPO concentration in processed whey. The column was then flushed with buffer solution A. Separation of LF and LPO was triggered by flushing the column using two buffer solutions (mixture of sodium citrate, phosphate, TRIS and carbonate buffers, 4 to 50 mM, pH=4.6 and 12.0) in linear pH gradient mode. The pH was gradually linearly changed in a range from 4.6 to 12.0. The pH ranges for LPO/LF elution were 8.9-10 and 11-11.7, respectively. The results of the separation procedure were two, chromatographically very well separated elution fractions of LPO and LF, which were further easily processed separately. In the following steps, fractions of the isolated proteins were neutralised to pH=6 using a small amount of appropriate acid solution, concentrated using TFF membrane with a pore size from 1 to 50 kDa and spray dried. Final LF and LPO purities were >98% and >70%, respectively. LF C- and A-values were determined to be 71% and 3.4%, respectively.

### Example 2

A monolith column, 80 mL CIMmultus™ SO3; BIA Separations was equilibrated before loading by using buffer solution B (sodium phosphate or citrate buffer: 5-50 mM, pH=5.0 to 6.5, as the pH of sweet whey). After that, sweet whey was allowed to flow through the column until the column capacity for LF and LPO reached. The saturation of the column capacity was verified by analysing flow-through samples on the outflow side of the column by HPLC method described in Analytics section. The volume of whey pumped through the column at a flow rate of 0.24 L/min was usually 10 to 40 L, which was mainly depending on LF/LPO concentration in processed whey. The column was then flushed with a buffer solution B. Separation of LF and LPO was triggered by flushing the column using two buffer solutions (mixture of sodium citrate, phosphate, TRIS and carbonate buffers, 4 to 50 mM, pH=5.0 and 12.0) in linear pH gradient mode. The pH is gradually linearly changed in a range from 5.0 to 12.0. The pH ranges for LPO/LF elution were 8.9-10 and 11-11.7, respectively. The results of the procedure mentioned above were two, chromatographically very well separated fractions of LPO and LF, which were further easily processed separately. In the following steps, fractions of the isolated proteins were neutralised to pH=6 using a small amount of appropriate acid solution, concentrated using TFF membrane with a pore size from 1 to 50 kDa and spray dried. Final LF/LPO purity was >98% or >70%. LF C- and A-values were determined to be 70.2% and 3.9%, respectively.

### Example 3

A monolith column, 8 L CIMmultus™ SO3—Strong CEX; Bia Separations, was before loading equilibrated by 40 to 80 L of buffer solution C (sodium phosphate or citrate buffer: 5-50 mM with addition of NaCl, pH=4.6 and conductivity of 15 mS/cm). After that, acid whey was allowed to flow through the column until the column capacity for LF and LPO were reached. The saturation of the column capacity was verified by analysing flow-through samples on the outflow side of the column by HPLC method described in Analytics section. The volume of whey pumped through the column at a flow rate of 8 L/min was usually 1000 to 2000 L, which was mainly depending on LF/LPO concentration in processed whey. The column was then flushed with buffer solution C. Separation of LF and LPO was conducted by flushing the column using two buffer solutions (mixture of sodium citrate, phosphate, TRIS and carbonate, 4 to 50 mM with addition of NaCl, pH=4.6 and 12.0) in linear pH gradient mode. The pH was gradually linearly changed in a range from 4.6 to 12.0, while conductivity (15 mS/cm) stayed constant trough whole linear pH gradient. The pH ranges for LPO/LF elution were 8.2-9.3 and 10.7-11.2, respectively. The results of the procedure mentioned above were two, the chromatographically very well separated elution fractions of LPO and LF, which were further easily processed separately. In the following steps, the fractions of collected proteins were first neutralised to pH=6 using a small amount of appropriate acid solution, desalted and concentrated by using TFF membrane with a pore size from 1 to 50 kDa and dried by spray drying. Final LF/LPO purity was >98% or >75%. LF C- and A-values were determined to be 74.2% and 2.5%, respectively.

### Example 4

A monolith column, 8 L CIMmultus™ SO3—Strong CEX; Bia Separations, was before loading equilibrated by using buffer solution C (sodium phosphate or citrate buffer: 5-50 mM, pH=5.0 to 6.5, as the pH of sweet whey). After that, acid whey was allowed to flow through the column until the column capacities for LF and LPO were reached. The saturation of the column capacity was verified by analysing flow through samples on outflow site of the column by HPLC method described in the Analytics section. The volume of whey pumped through the column at a flow rate of 8 L/min was usually 1000 to 2000 L, which was mainly depending on LF/LPO concentration in processed whey. Proteins with IEP <7.5 were initially eluted by pH step elution mode using a buffer solution C with pH=7.5 and then separation of LF and LPO was performed by flushing the column in linear pH gradient mode using same buffers as in Example 2. The pH was gradually linearly changed in a range from 7.5 to 12.0. The pH ranges for LPO/LF elution are 8.9-10 and 11-11.7, respectively. Separately collected elution fractions of LPO and LF were then neutralised to pH=6 using a small amount of appropriate acid solution, concentrated by using TFF membrane with a pore size from 1 to 50 kDa and dried by spray drying. Final LF and LPO purities were >98% and >75%, respectively. LF C- and A-values were determined to be 72.6% and 2.8%, respectively.

### Example 5

A monolith column, 8 L CIMmultus™ SO3—Strong CEX; Bia Separations, was before loading equilibrated by using buffer solution C (sodium phosphate or citrate buffer: 5-50 mM with addition of NaCl, pH=4.6 and conductivity of 15 mS/cm). After that, acid whey was allowed to flow through the column until the column capacities for LF and LPO were reached. The saturation of the column capacity was verified by analysing flow through samples on the outflow side of the column by HPLC method described in the Analytics section. The volume of the whey pumped through the column at a flow rate of 8 L/min was usually 1000 to 2000 L, which was mainly depending on LF/LPO concentration in processed whey. The column was then flushed with buffer solution C. Proteins with IEP <7.5 were initially eluted by pH step elution mode using a buffer solution C with pH=7.5 and then separation of LF and LPO was performed by flushing the column in linear pH gradient mode using same buffers as in Example 3. The pH was gradually linearly changed in a range from 7.5 to 12.0, while the conductivity (15 mS/cm) stayed constant through the whole pH gradient. The pH ranges for LPO/LF elution were 8.2-9.3 and 10.7-11.2, respectively. Separately collected elution fractions of LPO and LF were then neutralised to pH=6 using a small amount of appropriate acid solution, concentrated by using TFF membrane with a pore size from 1 to 50 kDa and dried by spray drying technique. Final LF and LPO purities were >98% and >80%, respectively. LF C- and A-values were determined to be 71.6% and 3.2%, respectively.

### Example 6

A monolith column, 8 L CIMmultus™ SO3—Strong CEX; Bia Separations, was before loading equilibrated by using buffer solution C (sodium phosphate or citrate buffer: 5-50 mM, pH=4.6). After that, acid whey was allowed to flow through the column until the column capacities for LF and LPO were reached. The saturation of the column capacity was verified by analysing flow through samples on the outflow side of the column by the HPLC method described in the Analytics section. The volume of the whey pumped through the column at a flow rate of 8 L/min was usually 1000 to 2000 L, which was mainly depending on LF/LPO concentration in processed whey. The column was then flushed with buffer solution C. Proteins with IEP <7.5 were initially eluted by pH step elution mode using a buffer solution C with pH=7.5 and then separation of LF and LPO was conducted by flushing the column using two buffer solutions (mixture of sodium citrate, phosphate, TRIS and carbonate, 4 to 50 mM with addition of NaCl to achieve appropriate conductivity, pH=4.6 and 12.0). The pH was gradually changed in a range from 7.0 to 12.0, while the gradient of conductivity increased from 4 to 55 mS/cm. The pH ranges for LPO/LF elution were 6.6-7.5 and 9.6-10.7, respectively. Separately collected elution fractions of LPO and LF were then neutralised to pH=6 using a small amount of appropriate acid solution, desalted and concentrated by using TFF membrane with a pore size from 1 to 50 kDa and dried by spray drying technique. Final LF and LPO purities were >98% or >85%. LF C- and A-values were determined to be 76.1% and 2.0%, respectively.

### REFERENCES

[1] Susana A. Gonzalez-Chavez, Sigifredo Arevalo-Gallegos, and QuintinRascon-Cruz. Lactoferrin: structure, function and applications. International Journal of Antimicrobial Agents, 33(4): 301.e1-301.e8, 2009.
[2] Nicolas Urtasun, Maria Fernanda Baieli, Daniela BelenHirsch, Maria Camila MartinezCeron, Osvaldo Cascone, and Federico Javier Wolman. Lactoperoxidase purification from whey by using dye affinity chromatography. Food and Bioproducts Processing, 103:58-65, 2017.
[3] Lin Chen, Chen Guo, Yueping Guan, and Huizhou Liu. Isolation of lactoferrin from acid whey by magnetic affinity separation. Separation and Purification Technology, 56(2):168-174, 2007.
[4] Rhee-M. S. Imm J Y Noh, K. H. Separation of lactoferrin from model whey protein mixture by reverse micelles formed by cationic surfactant. Food Sci. Biotechnol., 14:131-136., 2005.
[5] Shalan Alwan Al-Mashikhi, Eunice Li-Chan, and ShuryoNakai. Separation of immunoglobulins and lactoferrin from cheese whey by chelating chromatography. Journal of Dairy Science, 71(7):1747-1755, 1988.
[6] S. Yoshida, Z. Wei, Y. Shinmura, and N. Fukunaga. Separation of lactoferrin-a and -b from bovine colostrum. Journal of Dairy Science, 83(10):2211-2215, 2000.
[7] Jonatan Andersson and Bo Mattiasson. Simulated moving bed technology with a simplified approach for protein purification: Separation of lactoperoxidase and lactoferrin from whey protein concentrate. Journal of Chromatography A, 1107(1):88-95, 2006.
[8] Rong Rong Lu, Shi Ying Xu, Zhang Wang, and Rui Jin Yang. Isolation of lactoferrin from bovine colostrum by ultrafiltration coupled with strong cation exchange chromatography on a production scale. Journal of Membrane Science, 297(1):152-161, 2007.
[9] T. Uchida, S. Dosako, K. Sato, and H. Kawakami Sequential separation of lactoferrin, lactoperoxidase, and secretory component by sulfate-linked ion-exchange chromatography. Milchwissenschaft, 9(58):482-486, January 2003.
[10] O. Cascone Mariano Grasselli. Separation of lactoferrin from bovine whey by dye affinity chromatography. Netherlands Milk and Dairy Journal, 4(50):551-561, January 1996.
[11] Enrique Alvarez-Guerra and Angel Irabien. Extraction of lactoferrin with hydrophobic ionic liquids. Separation and Purification Technology, 98:432-440, 2012.
[12] Vojtech Adam, OndrejZitka, Petr Dolezal, Ladislav Zeman, Ales Horna, Jaromir Hubalek, Jan Sileny, Sona Krizkova, Libuse Trnkova, and Rene Kizek. Lactoferrin isolation using monolithic column coupled with spectrometric or micro-amperometric detector. Sensors (Basel), 1(8):464-487, January 2008.
[13] J. H. Nuijens and H. H. Van Veen. Isolation of lactoferrin from milk, Jan. 19 1999. U.S. Pat. No. 5,861,491.
[14] Conan J. Fee and Amita Chand. Capture of lactoferrin and lactoperoxidase from raw whole milk by cation exchange chromatography. Separation and Purification Technology, 48(2):143-149, 2006. Separation and Purification in the Food Industry.
[15] Chalore Teepakorn, Koffi Fiaty, and Catherine Charcosset. Comparison of membrane chromatography and monolith chromatography for lactoferrin and bovine serum albumin separation. Processes, 4(3), 2016.
[16] P. Milavec Zmak, H. Podgornik, J. Jancar, A. Podgornik, and A. Strancar. Transfer of gradient chromatographic methods for protein separation to convective interaction media monolithic columns. Journal of Chromatography A, 1006(1):195-205, 2003. International Symposium on Preparative and Industrial Chromatography and Allied Techniques.
[17] Shuichi Yamamoto and Ayako Kita. Theoretical background of short chromatographic layers: Optimization of gradient elution in short columns Journal of Chromatography A, 1065(1):45-50, 2005. 1st Monolith Summer School.
[18] Bo Wang, Yakindra Prasad Timilsena, Ewan Blanch, and Benu Adhikari. Characteristics of bovine lactoferrin powders produced through spray and freeze drying processes. International Journal of Biological Macromolecules, 95:985-994, 2017.
[19] K. Sato and S. I. Dousako. Process of separating, purifying and recovering milk proteins capable of binding iron, Mar. 27 1991. EP Patent App. EP19,900,117,442.
[20] K. Sato, M. Shiba, A. Shigematsu, N. Teduka, and A. Tomizawa. Method for producing lactoferrin, Oct. 13 2004. EP Patent App. EP20,040,008,008.
[21] G. Rowe, H. Aomari, and D. Petitclerc. New purification method of lactoferrin, November 16 2006. WO Patent App. PCT/CA2006/000,780.
[22] H. Kawakami, M Tanimoto, and S. Dousako. Process for separating and purifying lactoferrin from milk using sulfate compound, Sep. 9 1992. EP Patent 0,348,508.
[23] Z. Mozaffar, S. H. Ahmed, V. Saxena, and Q. R. Miranda. Sequential separation of whey, Aug. 1 2000. U.S. Pat. No. 6,096,870.
[24] J. Souppe. Milk protein isolate and process for its preparation, Dec. 10 2013. U.S. Pat. No. 8,603,560.
[25] Kussendrager K. D., Kivits M. G. C., and Verver A. B. Process for isolating lactoferrin and lactoperoxidase from milk and milk products, and products obtained by such process, Jun. 16 1998. CA Patent 2,128,111.
[26] S. Okonogi, M. Tomita, T. Tomimura, Y. Tamura, and T. Mizota. A process for producing bovine lactoferrin in high purity, Jul. 5 2006. EP Patent 0,253,395.
[27] K. D. Kussendrager, M. G. C. Kivits, and A. B. Verver. Process for isolating lactoferrin and lactoperoxidase from milk and milk products, and products obtained by such process, January 21 1997. U.S. Pat. No. 5,596,082.
[28] Jean-Paul Perraudin. Method for production of lactoferrin, August 2015. U.S. Pat. No. 9,115.211 B2.
[29] Kevin Neil Pearce Shaojiang Chen. Method of preparing low-iron lactoferrin, February 2012. EP2421894A1.
[30] Hassan Mohamed Hassan Abdalla. Method for purifying lactoferrin, December 2014. U.S. Pat. No. 9,458,225B2.
[31] Jan H. Nuijens Veen Harry H. Van. Isolation of lactoferrin from milk, August 1995. WO1995022258A2.
[32] Linda Voswinkel, Thomas Vogel, and Ulrich Kulozik. Impact of the iron saturation of bovine lactoferrin on adsorption to a strong cation exchanger membrane. International Dairy Journal, 56:134-140, 2016.
[33] Satoshi Ito, Katsuya Ikuta, Daisuke Kato, Kotoe Shibusa, Noriyasu Niizeki, Hiroki Tanaka, Lynda Addo, Yasumichi Told, Mayumi Hatayama, Junki Inamura, MotohiroShindo, Katsunori Sasaki, Naomi Iizuka, Mikihiro Fujiya, Yoshihiro Torimoto, and Yutaka Kohgo. Non-transferrin-bound iron assay system utilizing a conventional automated analyzer. ClinicaChimica Acta, 437:129-135, 2014.
[34] Robert J. Hilton, Matthew C. Seare, N. David Andros, Zachary Kenealey, Catalina Matias Orozco, Michael Webb, and Richard K. Watt. Phosphate inhibits in vitro Fe3+ loading into transferrin by forming a soluble Fe(iii) phosphate complex: A potential non-transferrin bound iron species. Journal of Inorganic Biochemistry, 110:1-7, 2012.

1. A method for manufacturing a fraction comprising the proteins lactoferrin and/or lactoperoxidase from a source containing at least one of these proteins wherein the source is selected from the group consisting of milk, colostrum, acid or sweet whey, by means of a chromatographic separation process with a monolithic column having strong cation exchanger properties, wherein in the separation process a pH gradient or a combined pH and salt gradient elution is employed after loading the source to the column.

2. The method of claim 1, wherein the pH gradient starts in a pH range of about 4.0 to about <pH 8.0.

3. The method of claim 1, wherein the pH gradient terminates in a range of about pH 8 to pH<13.

4. The method of claim 1, wherein the source is filtered prior to loading of the source to the column.

5. The method of claim 1, wherein a fraction A is collected which elutes at a pH range of about pH 8 to about pH<11, in particular about pH 8.9 to about pH 10 or about pH 6.6 to about pH 7.5 at a higher conductivity in the range about 5 to 55 mS/cm.

6. The method of claim 1, wherein a fraction B is collected which elutes at a pH range of about pH >10.4 to about 12, in particular about pH >11 to about 11.7, or about pH 9.6 to about pH 10.7 at a higher conductivity in the range about 5 to 55 mS/cm.

7. The method of the claim 1, wherein the chromatographic separation process comprises the steps of
(i) Adjusting the pH value of the source to a value lower than pH 7, in particular lower than pH 6.5;
(ii) Contacting the source of step (i) with a monolithic column having strong cation exchanger properties; followed by
(iii) Flowing a pH gradient buffer through the column thereby increasing the pH value; and
(iv) Collecting a fraction A which elutes at a pH range of about pH 8 to about pH<11, in particular about pH 8.0 to about pH 10, preferably at a pH range of about pH 8.2 to about pH 10, more preferably about pH 8.9 to about pH 10, or about pH 6.6 to about pH 7.5 at a higher conductivity in the range about 5 to 55 mS/cm and typically comprises lactoperoxidase; and/or
(v) Collecting a fraction B which elutes at a pH range of about pH >10 to about pH 12.0, preferably about pH >10.4 to about 12, more preferably about pH >11.0 to about pH 12.0, in particular about pH >11 to about 11.7, or about pH 9.6 to about pH 10.7 at a higher conductivity about 5 to 55 mS/cm and typically comprises lactoferrin;
(vi) Optionally further processing the fractions A and/or B, in particular by treatment for neutralising, concentrating, preservation and the like.

8. The method of claim 7, wherein the source is filtered prior to step (ii) or wherein the monolithic column is prior to step (ii) equilibrated with an equilibration buffer having a pH value of about pH<7, in particular about pH<6.5.

9. The method of claim 1, wherein the monolithic column having strong cation exchanger properties is selected from the group consisting of a —SO₃H modified monolithic column, —COOH modified monolithic column, —OSO₃H modified monolithic column or —OPO₃H modified monolithic column.

10. The method of claim 1, wherein the salt gradient is performed by concentration of salts, in particular the salt gradient corresponds to a conductivity in a range of about 5 mS/cm to about 55 mS/cm.

11. The method of claim 1, wherein prior to step (iii) or (iv) the column is flushed with the equilibration buffer of claim 8.

12. The method according to claim 1, wherein the lactoferrin and lactoperoxidase containing fractions are dried, in particular by spray drying.

13. The method according to claim 1, wherein the purity of lactoferrin is >90% and the purity of lactoperoxidase is >50%, in particular wherein the lactoferrin C value is >50 and the lactoferrin A value is >1.

14. The method according to claim 1, wherein the column is sanitised by flushing the column with a buffer of about pH >12 after step (iv) or (v).

15. A composition of matter comprising lactoferrin having a C value of >60% and A value of >1%.

16. The composition of matter according to claim 15 wherein the C value is 70% or more and the A value is 2% or more.

17. A composition of matter comprising lactoferrin or lactoperoxidase obtainable by a method according to claim 1.
